(19) United States
(12) Patent Application Publication
(10) Pub. No.: US 2026/0364292 A1
(43) Pub. Date: Nov. 19, 2026

(54) SPIRAL INTUBATION STYLET

(71) Applicant: East Carolina University, Greenville, NC (US)

(72) Inventor: Jason B. Hack, Greenville, NC (US)

(21) Appl. No.: 19/326,509

(22) Filed: Sep. 11, 2025

(51) Int. Cl. A61M 16/04; A61B 1/267

(52) U.S. Cl. CPC A61M 16/0418; A61B 1/267; A61M 2210/065

(57) ABSTRACT

An intubation stylet comprising a shaft having a proximal end and a distal end, wherein the shaft comprises an offset spiral configuration from the proximal end to the distal end; and a handle at the proximal end of the shaft, wherein the offset spiral configuration accommodates anatomical structures of a subject during an intubation. Intubation systems comprising the stylet and an endotracheal tube and methods of using the system are also disclosed.

### TECHNICAL FIELD

The presently disclosed subject matter relates to an intubation stylet and methods for using the stylet to intubate a subject.

### BACKGROUND

An intubation stylet is a device used to facilitate intubation of a patient. Referring to FIG. 1, shown is a conventional rigid stylet 10, which has a “hockey stick” configuration and is currently used when intubating a subject. During intubation, the stylet is inserted into the patient's throat along with an endotracheal tube and used to guide the endotracheal tube into the trachea of a subject. The configuration illustrated in FIG. 1 has been used since the 1940's, when practitioners were using direct laryngoscopy. “Hockey stick” stylet 10 includes a handle 20 at its proximal end 14 (closest to the user) of shaft 12 and a single, shallow bend 18 near its distal end 16 (farthest from the user). Proximal end 14 and distal end 16 of shaft 12 of stylet 10 are in the same plane, as viewed by the user upon an attempt to intubate a subject. This configuration does not accommodate the anatomy of a subject, and it can be very challenging for a medical practitioner, such as a physician, to intubate a subject efficiently using the stylet 10. This is particularly true for physicians or other medical practitioners in training, or who otherwise do not have extensive experience with intubation, or in patients with challenging anatomy. Yet, stylet 10 with its hockey stick configuration is still the only conformation commercially available for rigid stylets in use today.

What is needed, then, is an intubation stylet for intubation that is configured to accommodate subject's anatomical structures and to facilitate efficient intubation of a subject.

### SUMMARY

An intubation stylet is disclosed. In some examples, the intubation stylet comprises a shaft having a proximal end and a distal end, wherein the shaft comprises an offset spiral configuration from the proximal end to the distal end; and a handle at the proximal end of the shaft, wherein the offset spiral configuration accommodates anatomical structures of a subject during an intubation.

In some examples, the offset spiral configuration comprises a continuous curve that extends in three dimensions as the shaft runs from proximal end to distal end.

In some examples, the distal end of the shaft is in a different plane in a z dimension from the proximal end. In some examples, the distal end of the shaft is offset in one dimension from proximal end.

In some examples, the offset spiral configuration comprises a radius from a central point that accommodate anatomical structures of a subject during an intubation.

In some examples, the distal end of the stylet has an offset bend.

In some examples, the shaft comprises a rigid material. In some examples, the rigid material is a metal or a medically inert material, such as a plastic. In some examples, the metal is stainless steel.

In some examples, the handle at the proximal end of the shaft comprises a structure selected from the group consisting of a grip, a raised sleeve, a ring, a ball, a hook, a disk, a plunger, and any combination thereof. In some examples, the handle comprises a grip and a raised sleeve, wherein the raised sleeve is configured to receive an endotracheal tube.

In some examples, the handle comprises a moveable proximal endotracheal tube holder. In some examples, the moveable proximal endotracheal tube holder comprises a movable disk configured to contact an endotracheal tube. In some examples, the moveable proximal endotracheal tube holder comprises a movable disk and a plunger, wherein the movable disk is configured to contact an endotracheal tube and the plunger is configured to contact the movable disk.

A system for intubating a subject is disclosed. In one example, the system comprises an intubation stylet comprising a shaft having a proximal end and a distal end, wherein the shaft comprises an offset spiral configuration from the proximal end to the distal end; and a handle at the proximal end of the shaft, wherein the offset spiral configuration accommodates anatomical structures of a subject during an intubation; and an endotracheal tube disposed on the intubation stylet.

In some examples, a method of intubating a subject is disclosed. In some examples, the method comprises: inserting a laryngoscope into a mouth of the subject to visualize the subject's vocal cords within a larynx of the subject; inserting a system comprising: an intubation stylet comprising a shaft having a proximal end and a distal end, wherein the shaft comprises an offset spiral configuration from the proximal end to the distal end; a handle at the proximal end of the shaft, wherein the offset spiral configuration accommodates anatomical structures of a subject during an intubation; and an endotracheal tube disposed on the intubation stylet into a mouth of the subject; guiding the system to a trachea and through the vocal cords within the larynx of the subject using the laryngoscope; and deploying the endotracheal tube into the trachea of the subject.

In some examples, the laryngoscope is a video laryngoscope or is a direct laryngoscope.

In some examples, the handle comprises a moveable proximal endotracheal tube holder, which when extended causes a distal end of the endotracheal tube to extend beyond the distal end of the stylet, causing deployment of the endotracheal tube into the trachea of the subject.

In some examples, the moveable proximal endotracheal tube holder comprises a moveable disk at the proximal end of the shaft of the stylet in contact with the endotracheal tube. In some examples, sliding the moveable disk down a portion of the proximal end of the shaft of the stylet causes a distal end of the endotracheal tube to deploy beyond the distal end of the shaft of the stylet and into the trachea of the subject.

In some examples, the movable proximal endotracheal tube holder comprises a moveable disk and a plunger at the proximal end of the shaft of stylet, wherein the movable disk is in contact with the endotracheal tube and the plunger is in contact with the movable disk, and deploying the endotracheal tube comprises sliding the plunger down a portion of the stylet, causing deployment of the endotracheal tube to a trachea of the subject.

Accordingly, it is an object of the presently disclosed subject matter to provide an intubation stylet. This and other objects are achieved in whole or in part by the presently disclosed subject matter. Other objects will become evident as the description proceeds hereinbelow.

### BRIEF DESCRIPTION OF THE DRAWINGS

FIG. 1 is a side view of a conventional intubation stylet having a hockey stick configuration.

FIG. 2 is a front view of an exemplary intubation stylet of the presently disclosed subject matter.

FIG. 3 is a right side view of an exemplary intubation stylet of the presently disclosed subject matter.

FIG. 4 is a rear view of an exemplary intubation stylet of the presently disclosed subject matter.

FIG. 5 is a top rear view of an exemplary intubation stylet of the presently disclosed subject matter.

FIG. 6 is a top front view of an exemplary intubation stylet of the presently disclosed subject matter.

FIG. 7 is a schematic side cut away view of an exemplary endotracheal tube inserted into a subject.

FIG. 8 is a schematic side view of an approach taken for laryngoscopy using a video laryngoscope.

FIGS. 9 and 10 are schematic side views of an intubation of a subject using a video laryngoscope, and an exemplary intubation stylet of the presently disclosed subject matter with an endotracheal tube attached.

FIGS. 11-13 are a series of schematic side views of an anatomic cutaway to illustrate a patient's anatomy, a hockey stick stylet of the prior art being inserted into a patient's anatomy, and an exemplary intubation spiral stylet of the presently disclosed subject matter conforming to the patient's anatomy.

FIGS. 14A to 14C are schematic enlarged plan views of exemplary intubation stylets in accordance with the presently disclosed subject matter having alternative configurations at the proximal end of the shaft of stylet.

FIGS. 15A and 15B are schematic enlarged plan views of exemplary intubation stylets in accordance with the presently disclosed subject matter having alternative configurations at the proximal end of the shaft of stylet when in use to deploy the intubation stylet.

FIG. 16 is a front view of an exemplary intubation stylet of the presently disclosed subject matter including a ridged sleeve positioned just distal to the base of the grip of the handle.

### DETAILED DESCRIPTION

The presently disclosed subject matter will now be described more fully hereinafter with reference to the accompanying Figures, in which example embodiments are shown. The presently disclosed subject matter can, however, be embodied in different forms and should not be construed as limited to the example embodiments set forth herein. Rather, these example embodiments are provided so that this disclosure will be thorough and complete, and will fully convey the scope of the example embodiments to those skilled in the art.

All publications, patent applications, patents, and other references mentioned herein are incorporated by reference in their entirety.

### I. Definitions

While the following terms are believed to be well understood by one of ordinary skill in the art, the following definitions are set forth to facilitate explanation of the presently disclosed subject matter.

Unless otherwise defined, all technical and scientific terms used herein have the same meaning as commonly understood by one of ordinary skill in the art to which this presently described subject matter belongs.

Following long-standing patent law convention, the terms “a”, “an”, and “the” refer to “one or more” when used in this application, including the claims. Thus, for example, reference to “a pulse” or “a camera” includes a plurality of such pulses or cameras, and so forth.

Unless otherwise indicated, all numbers expressing quantities of time, pressure, distance, angle degree, weight, height, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a weight, temperature, pressure, angle, distance, depth, area, volume, percentage, time, rate, etc., is meant to encompass variations of in some examples ±20%, in some examples ±10%, in some examples ±5%, in some examples ±1%, in some examples ±0.5%, and in some examples ±0.1% from the specified amount, as such variations are appropriate to perform the disclosed methods or employ the disclosed devices or systems.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

### II. Intubation Stylet and Methods of Using the Same

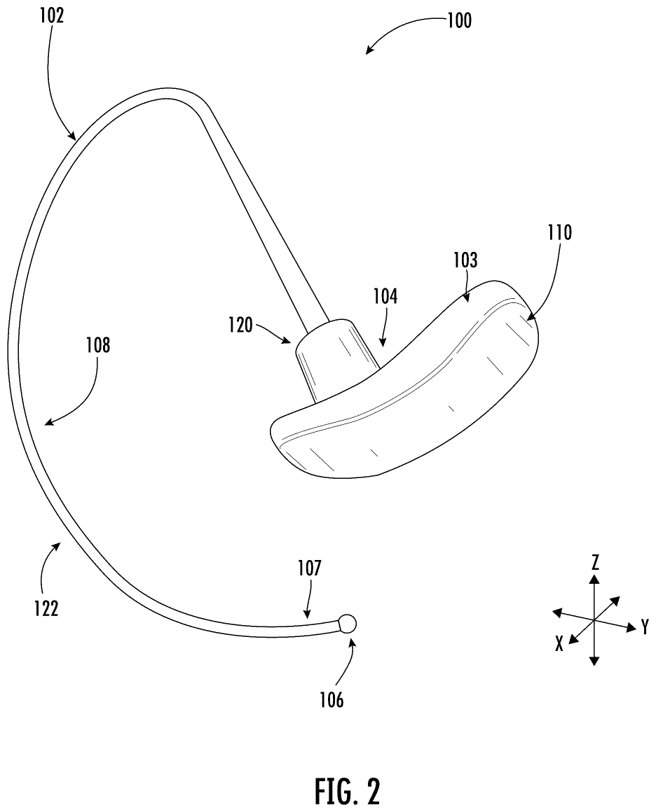

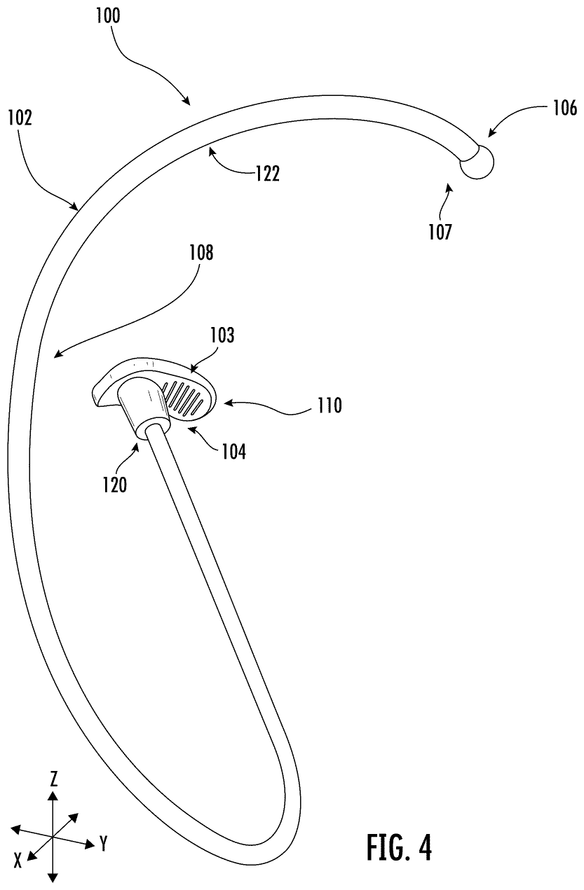

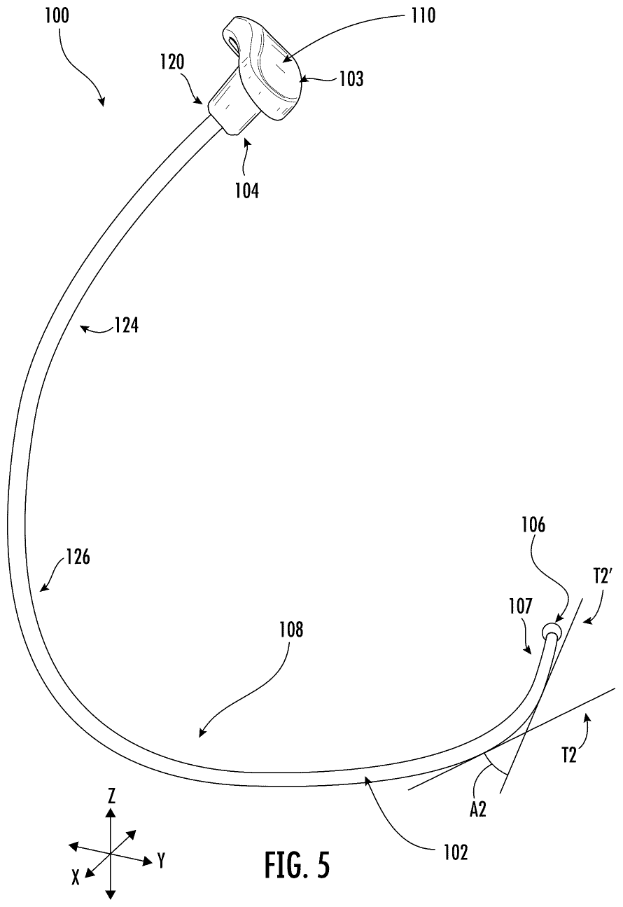

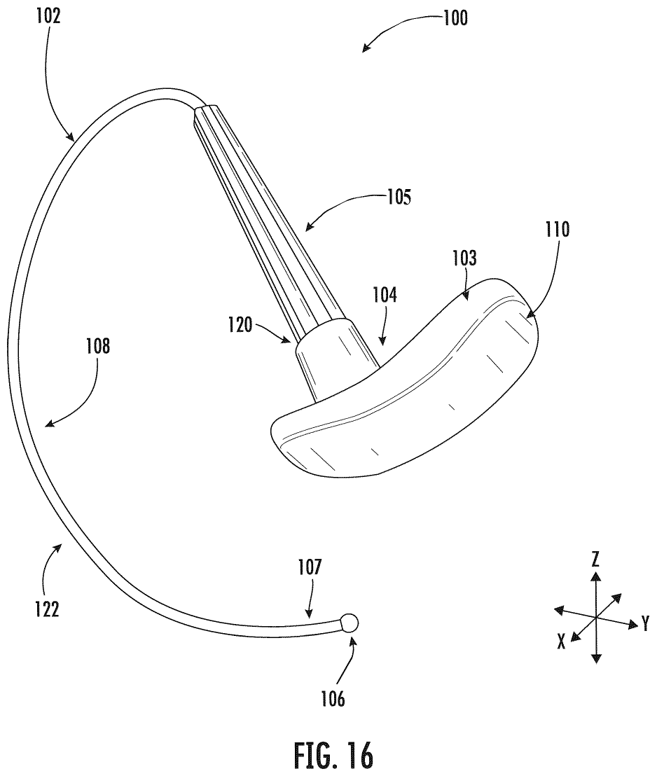

The presently disclosed subject matter provides, in some examples, a stylet device for use in the intubation of a subject. Referring now to the Figures, wherein like reference numerals refer to like parts throughout, an exemplary intubation stylet according to an example embodiment of the presently disclosed subject matter is referred to generally at 100. Referring to FIGS. 2-6 and 16, intubation stylet 100 comprises a shaft 102 having a proximal end 104 and a distal end 106. Shaft 102 comprises a spiral configuration 108 from proximal end 104 to distal end 106, wherein spiral configuration 108 accommodates anatomical structures of a subject during an intubation. Stylet 100 comprises a handle 110 at proximal end 104 of shaft 102. Handle 110 comprises grip 103 and in some examples a ridged sleeve 105 positioned just distal to base 120 of grip 103 (see FIG. 16). Sleeve 105 can aid in securing an endotracheal tube to stylet 100.

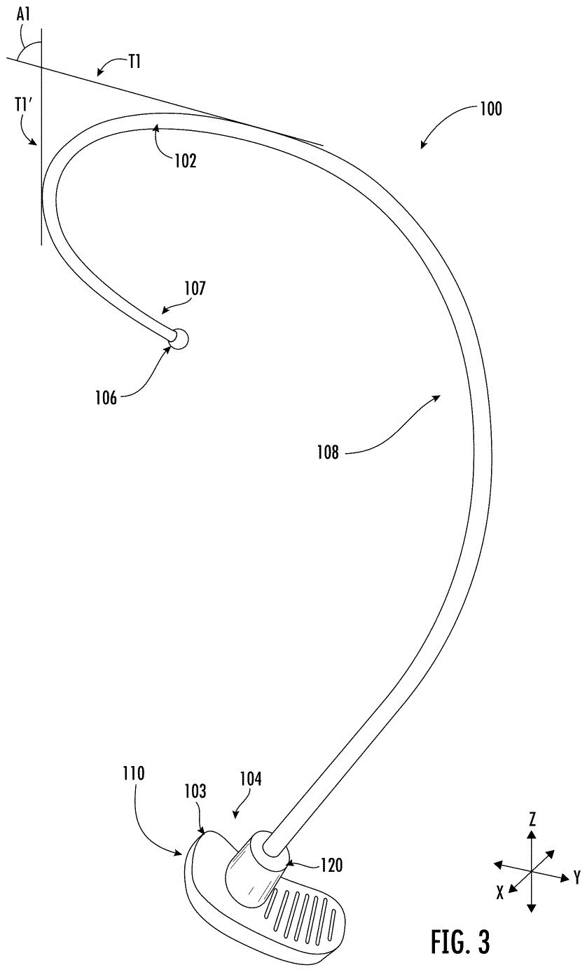

By way of example and not limitation, the configuration of shaft 102 can be formed by providing a straight shaft and bending it into a substantially circular shaft. Distal end 106 of shaft 102 is then bent into a direction, such as the z direction. Thus, continuing with reference to FIGS. 2-6 and 16, in some examples, spiral configuration 108 comprises a continuous curve that extends in in three dimensions (such as x, y, and z dimensions) as shaft 102 runs from proximal end 104 to distal end 106. In some examples, distal end 106 of shaft 102 is in a different plane from proximal end 104, as viewed by the practitioner upon an attempt to intubate a subject, such as in the z dimension. In some examples, in spiral configuration 108, distal end 106 of shaft 102 is offset in one dimension from proximal end 104, as viewed by the user upon an attempt to intubate a subject, as a result of the continuous curve in shaft 102. Thus, spiral configuration 108 can be referred to as an “offset spiral” or an “offset spiral configuration.” In some examples, handle 110 is positioned laterally as viewed by the practitioner upon an attempt to intubate a subject, such as about 90 degrees laterally from a plane of shaft 102 that would be inserted into a patient's mouth. Referring in particular to FIGS. 3, 5, and 6, in some examples, the continuous curve of shaft 102 can be further described in reference to pairs of intersecting tangent lines T1 and T1′, which define angle A1; intersecting tangent lines T2 and T2′, which define angle A2; and intersecting tangent lines T3 and T3′, which define angle A3. In some embodiments, angles A1, A2, and A3 can be the same or different. In some embodiments, each of angles A1, A2, and A3 range from about 40° to about 50°, including about 45°.

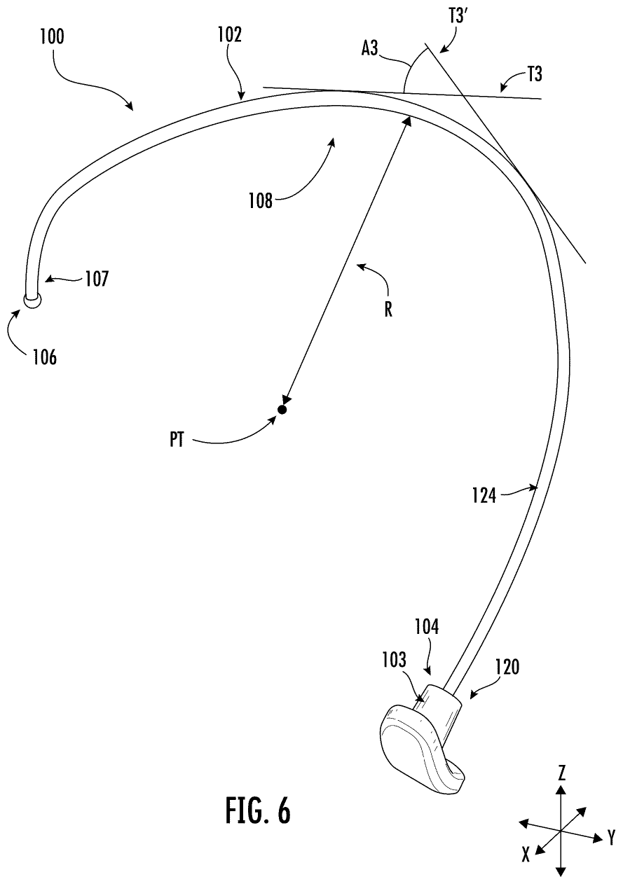

Continuing with reference to FIGS. 2-6 and 16, proceeding from handle 110, in spiral configuration 108, shaft 102 follows a continuous curve that travels through each of three dimensions (such as x, y, and z dimensions) from proximal end 104 of shaft 102 to distal end 106 with a radius R from a central point PT of the curve (see FIG. 6) that accommodates anatomical structures of a subject during an intubation. In some examples, radius R ranges from about 2 to about 3 inches. In some examples, shaft 102 includes a bend 122 and the width span from bend 122 to distal end 106 is about 3 inches. In some examples, the width span of shaft 102 from distal end 124 of sleeve 105 to distal end 106 of shaft 102 ranges from about 5 to about 7 inches. In some examples, the width span of shaft 102 from bend 126 of shaft 102 to distal end 106 of shaft 102 ranges from about 5 to about 6 inches. In some examples, distal end 106 of stylet 100 has an added offset bend 107 as compared to a portion of the shaft preceding it, or is in line with a portion of the shaft preceding it.

In some examples the intubation stylet comprises a medically inert rigid or semi-rigid material. In some examples, the rigid or semi-rigid material comprises a metal. In some examples, the metal is stainless steel. In some examples, the rigid or semi-rigid material comprises a plastic, a carbon-based material, or another material with similar properties.

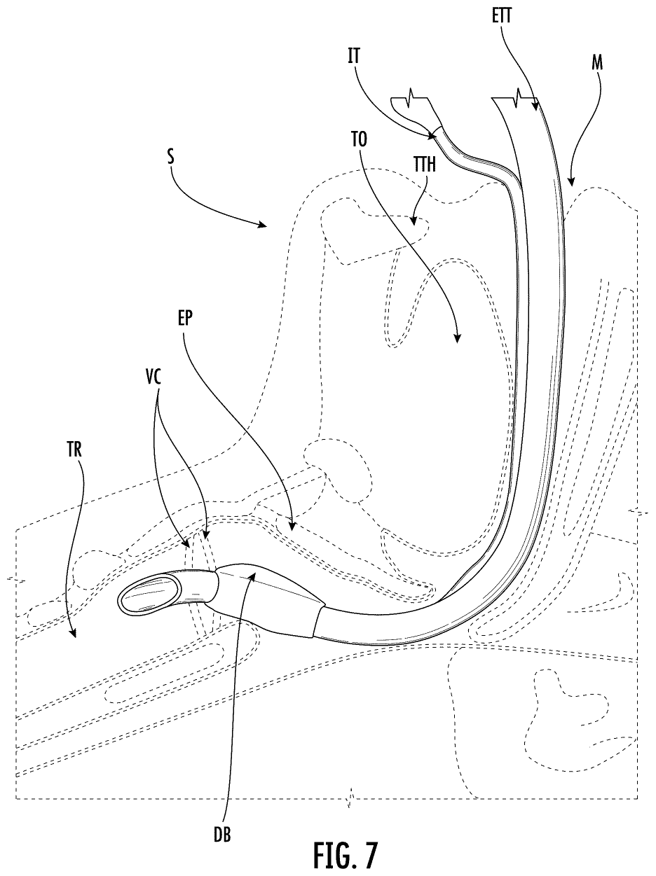

Referring to FIG. 7, a cutaway of a subject S (i.e., an intubated patient), showing the circuitous route that the flexible endotracheal tube ETT has to take to get from the opening of the mouth M past the lower teeth TTH, bend around the base of the tongue TO, slip under the epiglottis EP, and inserting into the trachea TR through and between the anatomically anteriorly located vocal cords VC. As can be seen in FIG. 7, all of this anatomy between the mouth opening and vocal cords VC causes a curve and potential impediments in the airway that need to be circumvented for the successful placement of endotracheal tube ETT. An inflation tube IT is also shown, which is used to inflate a distal balloon DB on endotracheal tube ETT as described herein. Endotracheal tube ETT is flexible, and its shape is determined solely by its modeling around the stylet that is inserted within it to make it hold a particular shape.

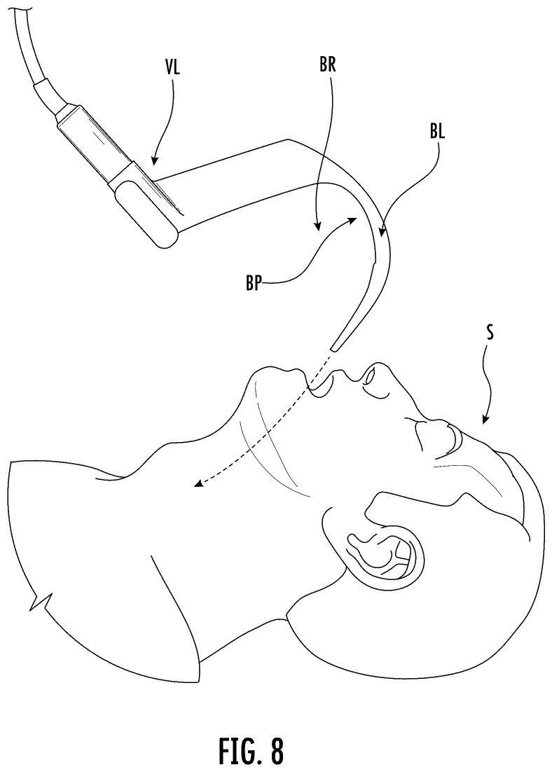

Referring to FIG. 8, laryngoscopy (visual examination of the larynx) with an example of a video laryngoscope is shown. There are many types and forms of video laryngoscopes available in the art, and any suitable video laryngoscope as would be apparent to one of ordinary skill upon a review of the instant disclosure can be employed. Video laryngoscopy was first used in the 2000's. A representative, non-limiting video laryngoscope VL in some forms includes a curved blade BL having a radius BR from a central point BP large enough so that is used to get around the soft tissues of a subject S (i.e., an intubation patient) during laryngoscopy (shown by dashed line with arrow in FIG. 8). Video laryngoscope VL includes a camera that is connected to a monitor, which is used by a practitioner to guide a procedure, such as an intubation procedure. An alternative approach to video laryngoscopy involves the use of a laryngoscope (e.g., a metal laryngoscope) for direct laryngoscopy. The typical metal laryngoscope has a blade with a distal light source. A practitioner uses this type of laryngoscope in order to create a straight visual axis from the practitioner's eyes, through the patient's mouth opening and down to the vocal cords within the larynx of the throat. It is inserted into the airway, to push the tongue and soft tissue out of the way, while the practitioner tries to look down into the airway and establish a straight visual axis to visualize the vocal cords of the larynx. Thus, from the practitioner's viewpoint, the laryngoscope goes down into the subject's mouth and is used to manipulate the soft tissues within the airway so that the practitioner can see down the subject's throat. Direct laryngoscopy and video direct laryngoscopy both use the hockey stick stylet conformation for endotracheal tube insertion.

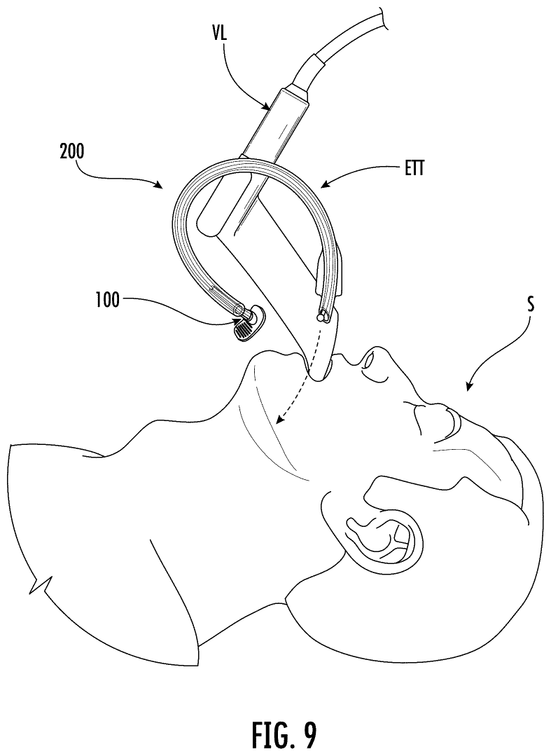

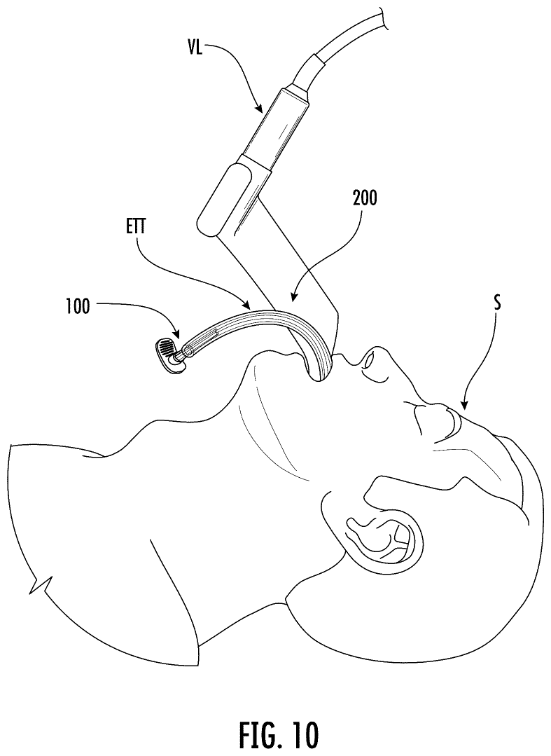

Referring now to FIGS. 9 and 10, intubation of a subject S (i.e., an intubation patient) using video laryngoscopy and an exemplary endotracheal tube stylet system in accordance with the presently disclosed subject matter is shown. Endotracheal tube ETT has been attached to stylet 100 to provide system 200. Any conventional endotracheal tube can be employed. The vocal cords are visualized using video laryngoscope VL. Any suitable video laryngoscope as would be apparent to one of ordinary skill in the art upon a review of the instant disclosure can be employed. Stylet 100 accurately conforms to the subject's anatomy and once it is inserted (shown by dashed line with arrow in FIG. 9) with endotracheal tube ETT, it accommodates anatomical structures of the subject and goes directly into the vocal cords within the larynx for an efficient and successful intubation. Thus, in some examples, system 200 leverages the advantages that video laryngoscopes provide.

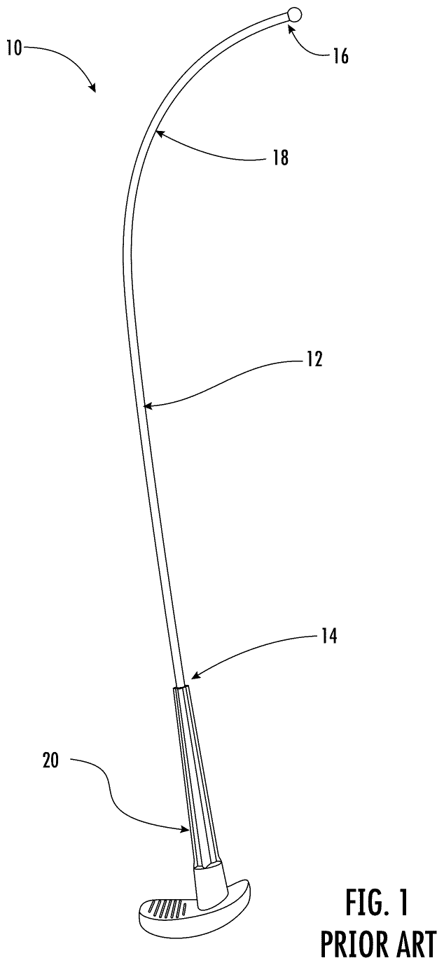

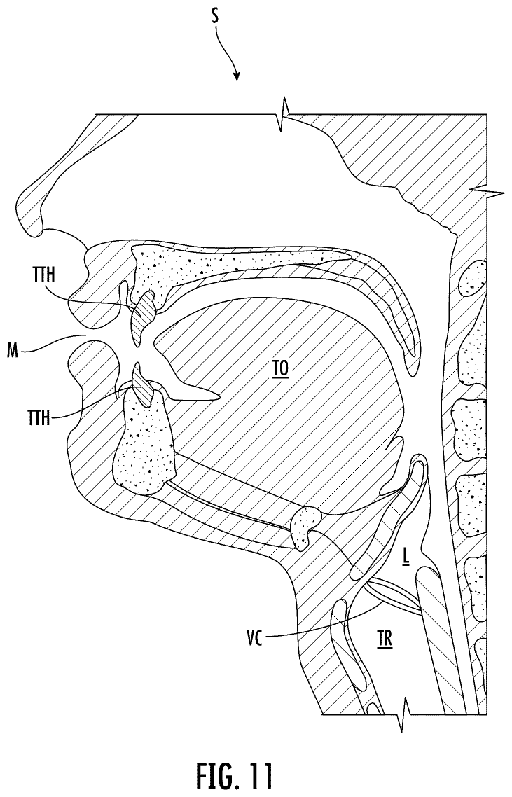

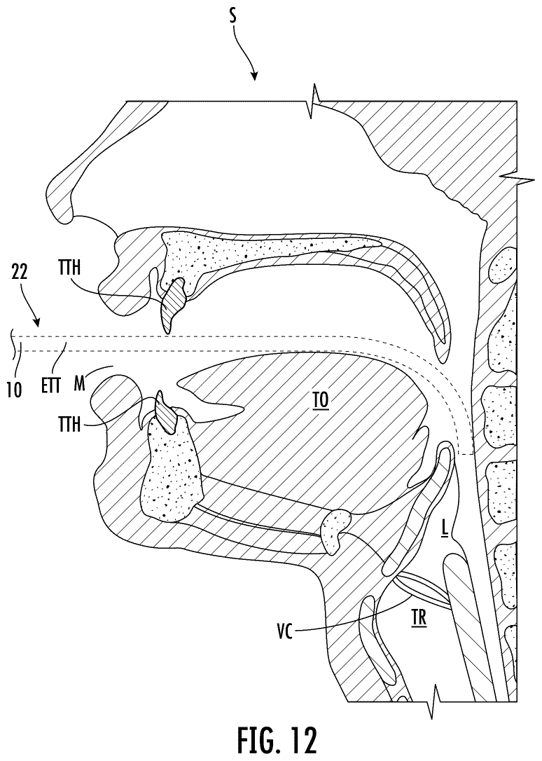

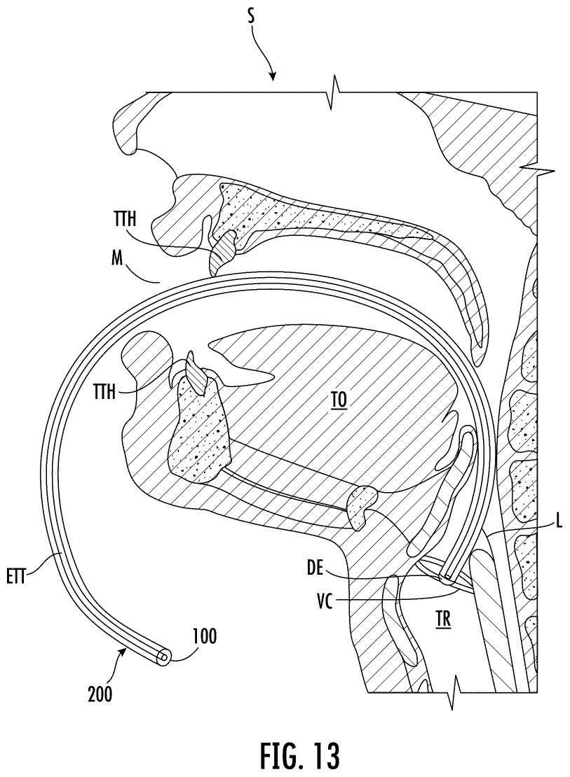

Referring to FIGS. 11 to 13, cut-away views of the airway anatomy of a human subject S are shown. Referring to FIG. 11, a cutaway of a subject S, showing the circuitous route that flexible endotracheal tube ETT has to take to get from the opening of mouth M past teeth TTH, around the base of tongue TO, through vocal cords VC (see also FIG. 7) of larynx L, and into trachea TR without an endotracheal tube stylet system. FIG. 12 shows in dashed lines an endotracheal tube stylet system 22 using the prior art hockey stick stylet 10 as shown in FIG. 1. As can be seen in FIGS. 11 and 12, all of this anatomy between mouth M and trachea TR causes a curve and potential impediments in the airway that need to be circumvented for the successful placement of endotracheal tube ETT. Referring to FIG. 13, exemplary system 200 in accordance with the presently disclosed subject matter accurately conforms to the subject's anatomy and accommodates anatomical structures of subject S so endotracheal tube ETT (i.e., distal end DE of endotracheal tube ETT) goes directly into trachea TR for an efficient and successful intubation.

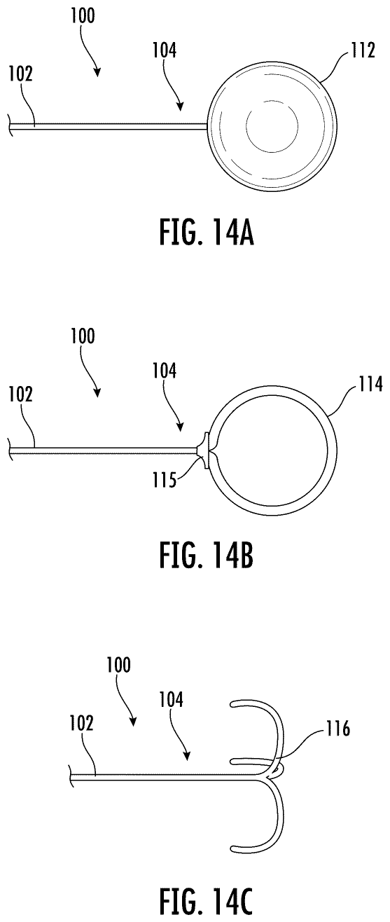

Referring to FIGS. 14A to 14C, exemplary endotracheal tube stylets in accordance with the presently disclosed subject matter can comprise alternative configurations at the proximal end of the shaft of stylet. In some examples, handle 110 at proximal end 104 comprises grip 103 and ridged sleeve 105 as shown in FIG. 16. In exemplary alternative embodiments, handle 110 can have an alternative configuration at proximal end 104. For example, a ball 112, ring 114, or hook 116 can be attached to or formed in shaft 102 at proximal end 104, with or without ridged sleeve 105. The practitioner can grab one of these structures to hold the stylet 100 while endotracheal tube ETT is being deployed.

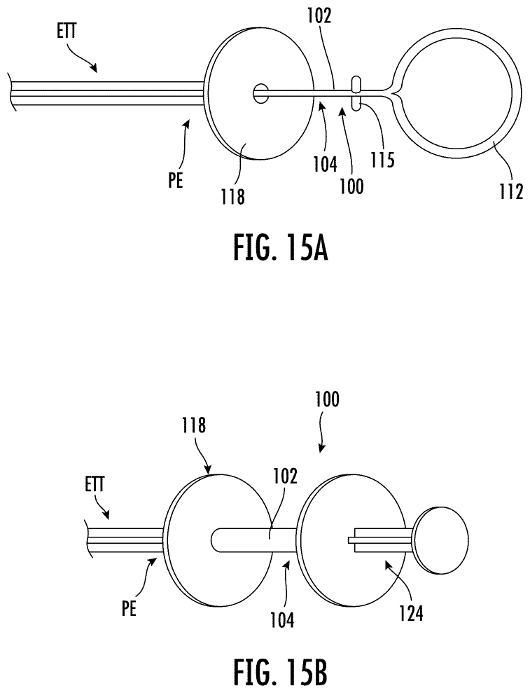

Continuing with reference to the Figures, in some examples, handle 110 comprises a moveable proximal endotracheal tube holder from which the endotracheal tube is extended from the distal end of the stylet, causing deployment (e.g., insertion) of the endotracheal tube into the trachea of the subject. For example, a moveable proximal endotracheal tube holder can comprise a moveable disk 118 at proximal end 104 of shaft 102 of stylet 100, for example, at base 120 of grip 103, instead of sleeve 105, or at base 115 of ring 114. Referring in particular to FIG. 15A, in some examples, stylet 100 is inserted into endotracheal tube ETT and proximal end PE of endotracheal tube ETT is slid up to the base 115 of ring 114 and engaged with disk 118 (in this example). During intubation, the practitioner uses a finger and slides disk 118 (in contact with endotracheal tube ETT) down a portion of the proximal end 104 of shaft 102 of stylet 100, causing distal end DE (see FIG. 13) of endotracheal tube ETT to extend beyond distal end 106 of shaft 102 of stylet 100 to cause deployment (e.g., insertion) of endotracheal tube ETT through the vocal cords VC within the larynx L off of distal end 106 of shaft 102 of stylet 100. In practice, the stylet typically and desirably does not enter the airway through the vocal cords VC of the larynx L. Rather, only the flexible endotracheal tube ETT enters the airway, and conforms and passes down the delicate trachea.

Referring now to FIG. 15B, in some examples, the presently disclosed stylet system comprises a moveable proximal endotracheal tube holder comprising a moveable disk 118 and a plunger 124 at the proximal end 104 of shaft 102 of stylet 100, instead of sleeve 105. Thus, in some examples, stylet 100 is inserted into endotracheal tube ETT and proximal end PE of the endotracheal tube ETT is slid up to disk 118, as in FIG. 15A. Plunger 124 engages with disk 118, in this example. During intubation, the practitioner moves/compresses plunger 124 and as a result, disk 118 (in contact with endotracheal tube ETT) slides down a portion of the proximal end 104 of shaft 102 of stylet 100, causing the distal end DE (see FIG. 13) of endotracheal tube ETT to extend beyond distal end 106 of shaft 102 of stylet 100 to cause deployment (e.g., insertion) of distal end DE (see FIG. 13) of endotracheal tube ETT through the vocal cords VC within the larynx L off of distal end 106 of shaft 102 of stylet 100. In practice, the stylet typically and desirably does not enter the airway through the vocal cords VC of the larynx L. Rather, only the flexible endotracheal tube ETT enters the airway, and conforms and passes down the delicate trachea.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. An intubation stylet comprising:
a shaft having a proximal end and a distal end, wherein the shaft comprises an offset spiral configuration from the proximal end to the distal end; and
a handle at the proximal end of the shaft, wherein the offset spiral configuration accommodates anatomical structures of a subject during an intubation.

2. The intubation stylet of claim 1, wherein the offset spiral configuration comprises a continuous curve that extends in three dimensions as the shaft runs from the proximal end to the distal end.

3. The intubation stylet of claim 1, wherein the distal end of the shaft is in a different plane in a z dimension from the proximal end.

4. The intubation stylet of claim 1, wherein the distal end of the shaft is offset in one dimension from the proximal end.

5. The intubation stylet of claim 1, wherein the offset spiral configuration comprises a radius from a central point that accommodate anatomical structures of a subject during an intubation.

6. The intubation stylet of claim 1, wherein the distal end of the shaft includes an offset bend.

7. The intubation stylet of claim 1, wherein the shaft comprises a rigid material.

8. The intubation stylet of claim 7, wherein the rigid material is a metal or a medically inert plastic.

9. The intubation stylet of claim 8, wherein the metal is stainless steel.

10. The intubation stylet of claim 1, where the handle at the proximal end of the shaft comprises a structure selected from the group consisting of a grip, a raised sleeve, a ring, a ball, a hook, a disk, a plunger, and any combination thereof.

11. The intubation stylet of claim 10, wherein the handle comprises a grip and a raised sleeve, wherein the raised sleeve is configured to receive an endotracheal tube.

12. The intubation stylet of claim 1, wherein the handle comprises a moveable proximal endotracheal tube holder.

13. The intubation stylet of claim 12, wherein the moveable proximal endotracheal tube holder comprises a movable disk configured to contact an endotracheal tube.

14. The intubation stylet of claim 12, wherein the moveable proximal endotracheal tube holder comprises a movable disk and a plunger, wherein the movable disk is configured to contact an endotracheal tube and the plunger is configured to contact the movable disk.

15. A system for intubating a subject, the system comprising:
an intubation stylet comprising a shaft having a proximal end and a distal end, wherein the shaft comprises an offset spiral configuration from the proximal end to the distal end;
a handle at the proximal end of the shaft, wherein the offset spiral configuration accommodates anatomical structures of a subject during an intubation; and
an endotracheal tube disposed on the intubation stylet.

16. A method of intubating a subject, the method comprising:
inserting a laryngoscope into a mouth of the subject to visualize the subject's vocal cords within a larynx of the subject;
inserting a system comprising: an intubation stylet comprising a shaft having a proximal end and a distal end, wherein the shaft comprises an offset spiral configuration from the proximal end to the distal end; a handle at the proximal end of the shaft, wherein the offset spiral configuration accommodates anatomical structures of a subject during an intubation; and an endotracheal tube disposed on the intubation stylet into a mouth of the subject; guiding the system to a trachea and through vocal cords of the subject using the laryngoscope; and
deploying the endotracheal tube into the trachea of the subject.

17. The method of claim 16, wherein the laryngoscope is a video laryngoscope or is a direct laryngoscope.

18. The method of claim 16, wherein the handle comprises a moveable proximal endotracheal tube holder, which when extended causes the distal end of the endotracheal tube to extend beyond the distal end of the stylet, causing insertion of the endotracheal tube into the trachea of the subject.

19. The method of claim 18, wherein the moveable proximal endotracheal tube holder comprises a moveable disk at the proximal end of the shaft of the stylet in contact with the endotracheal tube and inserting the endotracheal tube comprises sliding the disk down a portion of the proximal end of the shaft of the stylet, causing a distal end of the endotracheal tube to deploy beyond a distal end of the shaft of the stylet and into the trachea of the subject.

20. The method of claim 18, wherein the movable proximal endotracheal tube holder comprises a moveable disk and a plunger at the proximal end of the shaft of stylet, wherein the movable disk is in contact with the endotracheal tube and the plunger is in contact with the movable disk, and deploying the endotracheal tube comprises sliding the plunger down a portion of the stylet, causing insertion of the endotracheal tube to a trachea of the subject.
